(19) United States
(12) Patent Application Publication
(10) Pub. No.: US 2008/0292855 A1
(43) Pub. Date: Nov. 27, 2008

(54) METHODS OF DELIVERING FRAGRANCE USING ETHYLENE VINYL ACETATE RIBBON

(76) Inventors: Cary E. Manderfield, Racine, WI (US); Carl S. Weiss, Buffalo Grove, IL (US); John A. Schroeder, Racine, WI (US)

Correspondence Address: S.C. JOHNSON & SON, INC., 1525 HOWE STREET, RACINE, WI, 53403-2236, US

(21) Appl. No.: 11/751,201

(22) Filed: May 21, 2007

(51) Int. Cl. B32B 27/18; A61L 9/04

(52) U.S. Cl. 428/220; 512/4

(57) ABSTRACT

A substrate for delivering a fragrance to the surroundings is disclosed, wherein the substrate comprises: (i) at least one copolymer of an olefin and one or more ethylene derivatives selected from a group consisting of vinyl esters of saturated carboxylic acids, propenyl esters of saturated carboxylic acids, olefmically unsaturated organic acids or esters, and mixtures thereof, and (ii) a fragrance in an amount sufficient to be released to the surroundings at a sustained, controlled rate. Preferably, the substrate is in a form of a transparent sheet so that the substrate can be conveniently and discreetly attached to a surface and deliver a fragrance impregnated therein to the surroundings.

### BACKGROUND

1. Technical Field

A fragrance-impregnated copolymer strip or ribbon is disclosed for controlled delivery of the fragrance to the environment.

2. Description of the Related Art

Fragrances are molecules that diffuse via vaporization and activate one or more receptors in a user's nasal cavity. Often the level of fragrance needed in a product to achieve the desired response is relatively low. This is particularly true for a fragrance that has a high vapor pressure at room temperature and atmospheric pressure, and that has a high odor activity. In this disclosure, the terms “fragrance” and “perfume” will be used interchangeably and the singular terms, fragrance and perfume, may mean one or more different fragrant molecules such as blends or mixtures of fragrant molecules.

Malodors, on the other hand, are typically organic molecules with different structures and functional groups, such as amines, acids, alcohols, aldehydes, ketones, phenols, polycyclics, indoles, aromatics, polyaromatics, etc. Malodors can be treated in a variety of ways, one of which is the masking of their presence with another odor, such as a fragrance. Thus, odor masking can be defined as the intentional concealment of one odor (the malodor) with another (the fragrance). However, odor masking may require large quantities of fragrance to ensure that the malodor is no longer noticeable, or is suitably masked. Further, as fragrances do not chemically modify or neutralize malodor molecules, odor masking techniques require stable and prolonged delivery of fragrance while the malodor is present.

Controlled release of fragrance traditionally requires prepackaging of encapsulated fragrances into the final product. Although encapsulation can be functional in some cases, encapsulation can result in unbalanced fragrance release profiles due to either chemical incompatibility of the fragrance with the encapsulating agents or poor distribution of the fragrance in the encapsulated particles/substances. Moreover, encapsulation is expensive and therefore adds to the cost of the underlying product.

Some polymeric compositions, such as thermoplastics, can be impregnated with fragrances for controlled release of fragrance. However, while it would be desirable or even necessary to deliver fragrance discreetly, there is no currently commercially available clear or transparent polymeric substrate that can be impregnated with fragrance that can be discreetly placed where needed. Further, there is no currently available substrate that can be impregnated with fragrance and that does not require an additional support structure that would draw attention to the substrate. Further, currently available substrates do not provide a sufficient, controlled and prolonged fragrance delivery.

Therefore, there is a need for a convenient and discreet means for delivering fragrance to ambient surroundings in a sustained and controlled manner that is easy to manufacture from a cost effective material.

### SUMMARY OF THE DISCLOSURE

In satisfaction of the aforenoted needs, a substrate comprising a thermoplastic material and a fragrance impregnated therein is disclosed. The substrate is preferably in a form of a transparent sheet with optimized fragrance release characteristics. The sheet may be tinted or colored, depending upon the specific application.

According to one embodiment, the substrate comprises a thermoplastic copolymer with fragrance impregnated therein. In a refinement, the substrate is provided in the form of a transparent sheet having a thickness of less than 200 mils, and which achieves a 50% cumulative fragrance depletion in a time period ranging from about 5 to about 40 days, more preferably from about 10 to about 30 days, and even more preferably from about 10 to about 25 days.

According to another embodiment, the substrate comprises a thermoplastic copolymer with a fragrance impregnated therein, wherein the substrate is in a form of a transparent sheet having a thickness of less than 200 mil, and achieves an 8 mg/cm² cumulative fragrance emission rate of in a time period ranging from about 5 to about 40 days, more preferably from about 10 to about 30 days, and even more preferably from about 10 to about 25 days.

In a refinement, the substrate not only achieves 50% cumulative perfume depletion in a time period ranging from about 5 to about 40 days, but also achieves 8 mg/cm² cumulative fragrance emission rate of in a time period ranging from about 5 to about 40 days.

According to another embodiment, the substrate comprises a polymer matrix consisting essentially of a copolymer of an olefin and at least one olefin derivative selected from the group consisting of vinyl esters of saturated carboxylic acids, propenyl esters of saturated carboxylic acid, olefinically unsaturated organic acids or esters, and mixtures thereof. In a refinement, the olefin is ethylene. In another refinement, the vinyl ester is vinyl acetate. In yet another refinement, the propenyl ester is selected from the group consisting of 1-propenyl acetate, 2-propenyl acetate, and a mixture thereof. In a preferred embodiment, the copolymer is ethylene vinyl acetate. In another embodiment, the copolymer is propylene vinyl acetate.

In one embodiment, the substrate comprises a fragrance that is controllably released to the surroundings for masking at least one malodor. In a refinement of this concept, the amount of the fragrance released to the surroundings is effective to substantially mask the at least one malodor. In another refinement of this concept, the amount of fragrance released to the surroundings is effective to give a pleasant smell that is perceived by a consumer.

In one embodiment, the substrate further comprises an adhesive layer attached to one surface of the polymer matrix. In a refinement, the adhesive layer is sandwiched between the polymer matrix and a release liner.

According to one aspect of this disclosure, the substrate is provided in the form of a sheet having a thickness ranging from about 0.1 to about 200 mils. In an embodiment, the sheet is translucent or transparent.

Methods of using the substrate for delivering a fragrance to the surroundings are also disclosed. In one embodiment, the substrate is attached to a surface by an adhesive layer coated onto the substrate. The fragrance is allowed to be released from the substrate in a controlled and prolonged manner. In another embodiment, the substrate is coated with a layer of adhesive and can be applied directly on a surface or an article. In a refinement, the substrate is transparent and therefore its presence on the surface or article to which it was attached is discreet.

Methods of manufacturing the substrate are also disclosed. According to one embodiment, the copolymer is dissolved in an organic solvent and mixed with the fragrance to form a homogeneous mixture. The mixture is later processed into a film by extrusion or casting followed by drying. In a refinement, the film is formed by casting the mixture to provide higher porosity thereof. In another embodiment, the copolymer is processed into a film by extruding or casting followed by drying. A mixture of fragrance and solvent or fragrance alone is then provided to the copolymer film by techniques well known in the art, such as by coating, absorption, adsorption, and the like.

Other advantages and features of the disclosed substrates and methods will be described in greater detail below. Although only a limited number of embodiments are disclosed herein, different variations of the embodiments would be apparent to those skilled in the art.

### BRIEF DESCRIPTION OF THE DRAWINGS

For a more complete understanding of the disclosed solutions and methods, reference should now be made to the accompanying drawings, wherein:

FIG. 1 graphically illustrates the cumulative fragrance depletion of a first fragrance-impregnated substrate versus time, particularly illustrating the fragrance depletion (Dₙ) of the substrate disposed in a chamber at room temperature and a like substrate disposed in a conventional room environment at room temperature;

FIG. 2 graphically illustrates the cumulative fragrance depletion (Dₙ) of a second fragrance-impregnated substrate versus time, particularly illustrating the fragrance depletion of the substrate disposed in a chamber at room temperature;

FIG. 3 graphically illustrates the cumulative fragrance emission rate (Eₙ) of a first fragrance-impregnated substrate, particularly illustrating the fragrance depletion of the substrate disposed in a chamber at room temperature and a like substrate disposed in a conventional room environment at room temperature;

FIG. 4 graphically illustrates the cumulative fragrance emission rate (Eₙ) of a second fragrance-impregnated substrate, particularly illustrating the fragrance depletion of the substrate disposed in a chamber at room temperature.

It should be understood, of course, that this disclosure is not limited to the particular embodiments and examples illustrated herein.

### DETAILED DESCRIPTION OF THE PRESENTLY PREFERRED EMBODIMENTS

In one embodiment, a substrate is used for delivering a fragrance to the surroundings, wherein the substrate comprises: (i) at least one copolymer of an olefin and one or more olefin derivatives selected from the group consisting of vinyl esters of saturated carboxylic acids, propenyl esters of saturated carboxylic acid, olefinically unsaturated organic acids and esters, and mixtures thereof, and (ii) a fragrance in an amount sufficient to be released to the surroundings at a sustained, controlled rate.

In another embodiment, a substrate is used for delivering a fragrance for masking malodors in the surroundings, wherein the substrate comprises: (i) at least one copolymer prepared from a mixture comprising an olefin and at least one olefin derivative selected from the group consisting of vinyl esters of saturated carboxylic acids, propenyl esters of saturated carboxylic acids, and mixtures thereof, and (ii) a fragrance in an amount that masks at least a substantial portion of the malodor.

In another embodiment, the substrate is substantially free of any homopolymers such as polystyrene, polyethylene, polypropylene, polybutene, polybutadiene, poly vinyl alcohol, polymethacrylate, polymethylmethacrylate, polyvinyl chloride, polyvinylacetate, and other conventional homopolymeric thermoplastics known in the art.

In yet another embodiment, the substrate is in the form of a sheet having a thickness of from about 1 to about 200 mils. In a refinement, the sheet is translucent or transparent.

In another embodiment, the substrate may comprise a mixture of from about 50 to about 99 wt % copolymer, and from about 1 to about 50 wt % fragrance. In yet another embodiment, the substrate may comprise from about 60 to about 90 wt % copolymer, and from about 10 to about 40 wt % fragrance. The substrate may also comprise from about 70 to about 80 wt % copolymer, and from about 20 to about 30 wt % fragrance.

In another embodiment, the substrate may comprise a polymer matrix made entirely of a copolymer. In a refinement, the copolymer is prepared from a mixture of ethylene and a vinyl ester. In another refinement, the polymer matrix is made entirely of one or more ethylene vinyl acetate copolymers.

According to one aspect of this disclosure, the substrate may be formed into films having a thickness ranging from about 0.1 to about 200 mils, preferably from about 10 to about 50 mils, more preferably from about 10 to about 30 mils, and even more preferably from about 15 to about 20 mils. However, it is to be understood that the thickness of the film is variable and should not be considered limiting to the scope of this disclosure. Films having a thickness of less than 0.1 mil or more than 200 mils may also be suitable for use in this disclosure, as long as the thickness of the film does not substantially affect the transparency and/or the fragrance delivery characteristics of the film.

Copolymer

The copolymer which is utilized in the substrates may be copolymers derived from a mixture of: (a) an olefin; and (b) at least one copolymerizable monomer selected from the group consisting of vinyl esters of saturated carboxylic acid, propenyl esters of saturated carboxylic acid, olefmically unsaturated organic acids and esters, and mixtures thereof. In one embodiment, the copolymer comprises the copolymerization product obtained from a mixture comprising from about 40 to about 80 wt % olefm, and from about 5 to about 60 wt % copolymerizable monomer. In another embodiment, the copolymer is prepared from a mixture comprising from about 55 to about 76 wt % olefm, and from about 10 to about 35 wt % copolymerizable monomer. Other variations of the composition of the copolymer will be apparent to those skilled in the art.

In one embodiment, the copolymer is prepared from mixtures comprising an olefin and at least one monomer selected from a group consisting of acrylic acid, acrylic ester, methacrylic acid, and methacrylic ester. In yet another embodiment, the copolymer is prepared from a mixture comprising an olefin and at least one vinyl or propenyl ester of a saturated carboxylic acid. In a preferred embodiment, the copolymer is prepared from a mixture comprising an olefin and an ester selected from the group consisting of vinyl acetate, 1-propenyl acetate, 2-propenyl acetate, and mixtures thereof.

The olefins utilized in preparing the copolymers generally are alpha olefins containing, for example, from 2 to about 6 carbon atoms. Examples of such olefins include ethylene, propylene, 1-butene, etc. In one embodiment, the olefin is ethylene.

The olefinically unsaturated organic acids or esters useful as the copolymerizable monomers include unsaturated mono- and di- carboxylic acids containing from about 3 to about 20 carbon atoms, and esters of such unsaturated mono or dicarboxylic acids. Alkyl esters of such unsaturated organic mono and di- carboxylic acids are particularly useful, and the alkyl group may contain from 1 to about 10 or more carbon atoms. In one embodiment, the alkyl group is an n-alkyl group. In yet another embodiment, the unsaturated organic carboxylic esters are mono carboxylic acid esters such as alkyl acrylates wherein the alkyl group is an n-alkyl group containing from 1 to about 10 carbon atoms. Examples of such acrylates include methyl acrylate, methyl methacrylate, propyl acrylate, n-butyl acrylate, n-butyl methacrylate, hexyl acrylate, etc.

The vinyl or propenyl esters which can be utilized in the formation of useful copolymers include vinyl or propenyl esters of saturated carboxylic acids. In one embodiment, the acid group utilized to form the vinyl or propenyl esters contains from 1 to 18 carbon atoms. Specific examples of useful vinyl and propenyl esters include vinyl acetate, 1-propenyl acetate, 2-propenyl acetate, vinyl propionate, 1-propenyl propionate, 2-propenyl propionate, vinyl hexanoate, 1-propenyl hexanoate, 2-propenyl hexanoate, vinyl neodecanoate, 1-propenyl neodecanoate, 2-propenyl neodecanoate, etc. In a preferred embodiment, the vinyl or propenyl ester is vinyl acetate.

The copolymers can be prepared by procedures well known to those skilled in the art. In one embodiment, the olefin and the copolymerizable monomer are fed in a continuous manner to a stirred pressure vessel. Free-radical initiator is fed into the vessel by a separate line. The flow rate of monomer is adjusted to provide constant molar ratios, while the feed rate is the same as the rate of discharge of polymer product and unpolymerized monomer from the reactor. Allowance is made for the different rates of copolymerization of the various copolymerizable monomers. The polymerization initiator may be a commercial peroxide, and a small amount of a telogen (chain transfer agent) also may be introduced into the reactor with the feed monomers to control the molecular weight.

The copolymer based on ethylene and vinyl acetate, i.e., ethylene vinyl acetate (EVA) is available from DuPont under the general trade designation Elvax®. For example, Elvax® 260 is an ethylene-vinyl acetate copolymer containing about 28 wt % vinyl acetate. Further examples of such polymers available from DuPont include Elvax®360, Elvax® 450, and Elvax® 650Q.

In addition to the above described copolymers, the substrate, may, in some embodiments, contain other active material or adjuvant to enhance the appearance, physical characteristics and process characteristics of the substrate. Preferably, such material or adjuvant is not a homopolymeric substance, and is only included in the composition at a relatively low level, preferably less than about 10% of the total weight of the substrate. Suitable active materials or adjuvants that may be included in the solid composition will be apparent to those skilled in the art.

Fragrance

As is well known, a fragrance normally consists of a mixture of different fragrant materials. The number of fragrant materials may be ten or more and range of fragrant materials used may vary. The materials come from a variety of chemical classes, but in general are water-insoluble oils. The molecular weight of a fragrance material may be in excess of 150, but should not exceed 300.

The fragrance included in the substrate may be present in an amount that is sufficient to deliver a pleasant smell that can be perceived by a consumer. In the presence of a malodor, the perfume included in the substrate may be present in an amount that masks at least a substantial portion of the malodor in the surroundings. The perfume included in the substrate is preferably present in an amount that not only completely masks the odor associated with the antimicrobial/deodorant agent, but also delivers a pleasant smell to be perceived by a consumer. In one embodiment, the perfume is present in the substrate in an amount of from about 1 to about 50 wt %, more preferably from about 10 to about 40 wt %, especially from about 20 to about 30 wt %. The amount of the perfume that is needed to mask malodors, and/or the amount of the perfume to deliver a pleasant smell to be perceived by a consumer would be apparent to those skilled in the art.

The perfume may comprise one or more fragrant materials or materials that provide chemically active vapors. In one embodiment, the perfume can comprise and/or include volatile, fragrant compounds including, but not limited to natural botanic extracts, essences, fragrance oils, synthetic fragrant materials and so forth. As is known in the art, many essential oils and other natural plant derivatives contain large percentages of highly volatile scents. In this regard, numerous essential oils, essences, and scented concentrates are commonly available from companies in the fragrance and food businesses. Exemplary oils and extracts include, but are not limited to, those derived from the following plants: almond; amyris; anise; armoise; bergamot; cabreuva; calendula; canaga; cedar; chamomile; coconut; eucalyptus; fennel; jasmine; juniper; lavender; lemon; orange; palm; peppermint; quassia; rosemary; thyme; and mixtures thereof.

Many fragrances have colors associated with the fragrance. For example, the color lavender or purple is often associated with a lavender scent; the color yellow is often associated with a chamomile scent, a daffodil and the like; the color red is often associated with a rose scent and the like; and the color green is often associated with the scents of aloe, wintergreen and the like. Accordingly, the color of the substrate may be selected to create a mental association between the user of the device and the agent that is capable of being sensed by the user through olfaction. Other color and scent combinations may be utilized.

As stated above, the sensing of the perfume through olfaction can be used to help induce a desired psychological state including, for example, states of relaxation, peace, sensuality, energy, visualization of specific times and/or places, spirituality, visualization of various natural settings, etc.

Method of Use

The substrate may be processed into one of many physical forms, including, but are not limited to, ribbon, film, sheet, block, tube, ring, band, or other shapes and forms that is suitable for the application of the substrate. Preferably, the substrate is substantially self-supportive. The substrate may be opaque, translucent or transparent. Moreover, the substrate may have a distinct color or colorless.

In one embodiment, the substrate may be attached to a surface or an article by a bonding material. The bonding material may be coated on one surface of the article, formed as a part of the article, or combined with the article to form a laminate. The quantity and type of bonding material may be based on the properties that are desired within particular commercial products and their applications.

Some example materials that may be used for the bonding material include pressure-sensitive adhesives (PSAs), hotmelt adhesives, reactive adhesives (e.g., isocyanates, cyanoacrylates, acrylics, ethylvinylacetates), latex adhesives and epoxies. The type of bonding material will be selected based on processing parameters, solubility and whether the bonding material forms part of the substrate.

In one embodiment, the substrate is self-adhesive and no additional bonding material is needed.

In another embodiment, bonding material is sandwiched between the substrate and a disposable release liner for easy application of the substrate on a surface. A consumer simply detaches the disposable release liner and attaches the substrate to desired surfaces through the bonding material.

The substrate may be attached to a surface or an article using other approaches, so long as the substrate remains suitably affixed or connected to the surface. Suitable means to affix the substrate to the surface or article may include any suitable fasteners, such as adhesive fasteners, cohesive fasteners, mechanical fasteners, or the like. In one embodiment, the fastening system includes mechanical fastening elements for improved performance. Suitable mechanical fastening elements can be provided by interlocking geometric-shaped materials, such as hooks, loops, bulbs, mushrooms, arrowheads, balls on stems, male and female mating components, buckles, snaps, or the like.

Method of Manufacturing

According to one embodiment, the substrate film may be prepared by preparing a homogeneous mixture comprising the copolymer, the optional adjuvant, the perfume, and a suitable solvent (such as toluene), and subsequently casting or coating the mixture onto a silicone release liner, followed by drying at an elevated temperature to remove the solvent. After drying, the film can be retained on the release liner, or the film can be removed from the release liner for use in the intended applications as described herein. Alternatively, the films can be prepared by melt extrusion of a dry blend of the copolymer with the above described perfume. The films may be used as produced (i.e., not oriented) or the films may be monoaxially or biaxially oriented by stretching at an elevated temperature followed by annealing (heat setting) at a higher temperature. Such orientation procedures are known to those skilled in the art.

When the copolymer and the optional adjuvant are not mutually soluble in a solvent, separate solutions of the copolymer and optional adjuvant are prepared in suitable organic solvents, and the solutions are then blended before the perfume is added. The solvent can be subsequently removed.

In one embodiment, there appears to be an advantage in preparing the substrate by solvent casting onto a release liner followed by drying to form the substrate. Substrates prepared in this manner contain an evenly distributed perfume impregnated therein, which facilitates the sustained controllable release of the perfume to the surroundings. In general, improved release of the perfume is observed for such substrate when compared to similar substrate formed by extrusion.

Non-limiting examples of coating techniques include slot die, air knife, brush, curtain, extrusion, blade, floating knife, gravure, kiss roll, knife-over blanket, knife-over roll, offset gravure, reverse roll, reverse smoothing roll, rod and squeeze roll coating. The application of the bonding material to the substrate can be carried out at room temperature or at elevated temperatures; and the substrate may be subjected to higher temperatures to accelerate evaporation of the solvents. Temperatures as high as 150° C. have been found to be useful.

Fragrance Delivery Characteristics

One important feature of the substrate is its ability to deliver the fragrance impregnated therein to the surroundings in a sustained, controlled manner.

For a sustained fragrance delivery, the substrate is capable of continuously delivering an effective amount of fragrance for human sensory satisfaction and/or malodor masking over an extended period of time. In one embodiment, the substrate has a sustained perfume delivery of at least about 7 days. In another embodiment, the substrate has a sustained fragrance delivery of at least about 22 days. In yet another embodiment, sustained fragrance delivery continues for at least about 36 days.

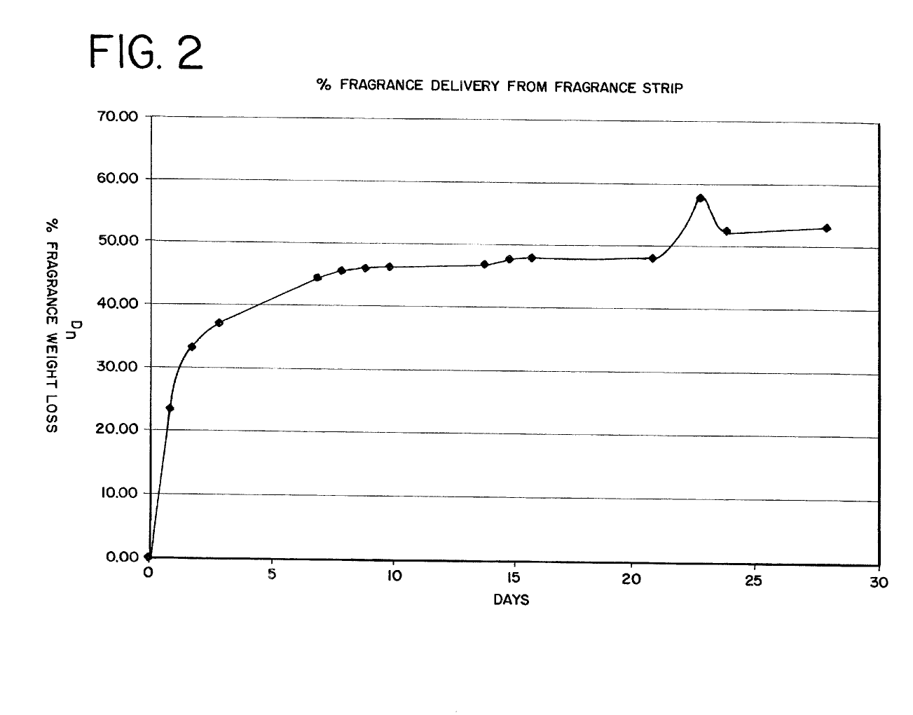

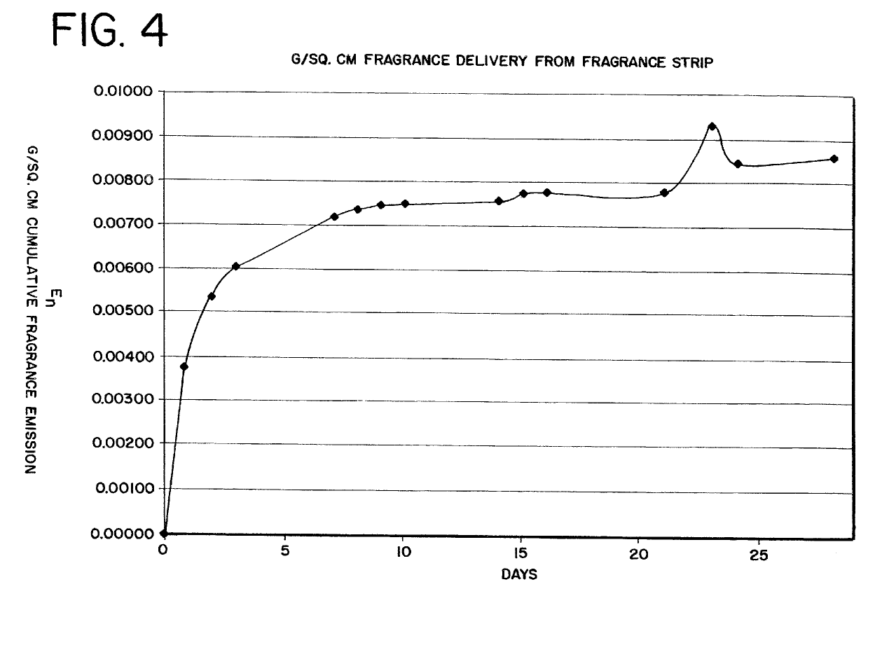

For a controlled fragrance delivery, the substrate should be capable of delivering the fragrance at a rate that is suitable for human sensory satisfaction and/or malodor masking. In one embodiment, fragrance is delivered in two stages, a first stage of rapid delivery during the first few days followed by a second stage of near steady-state delivery thereby keeping the fragrance concentration at a relatively stable level. This two-stage delivery is illustrated in FIGS. 2 and 4, wherein the fragrance is delivered at a relatively high rate during the first three days and a substantially slower but linear rate for the next seven days.

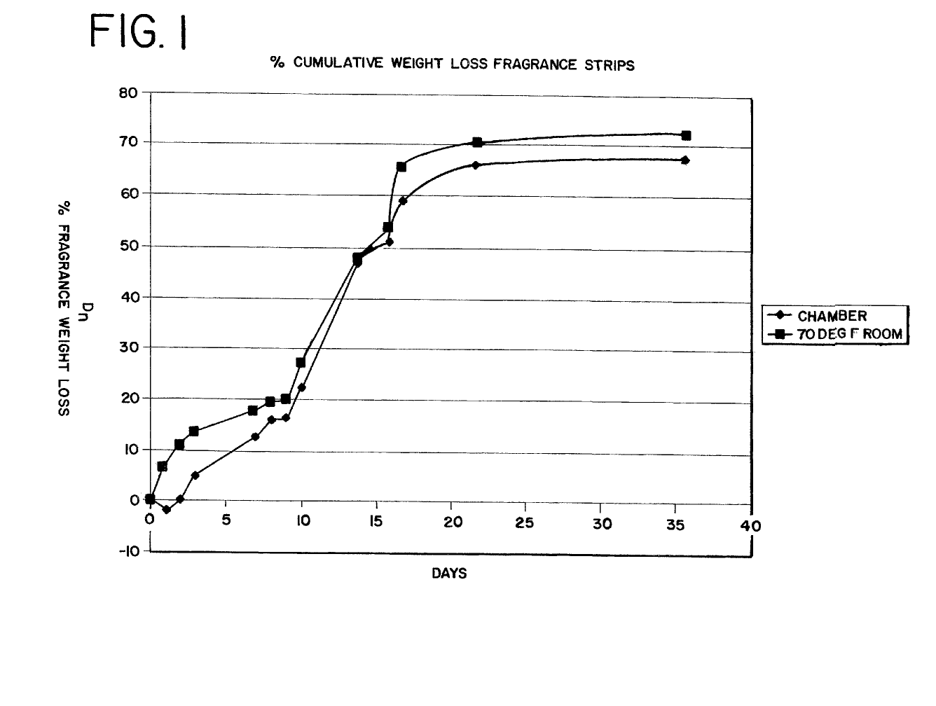

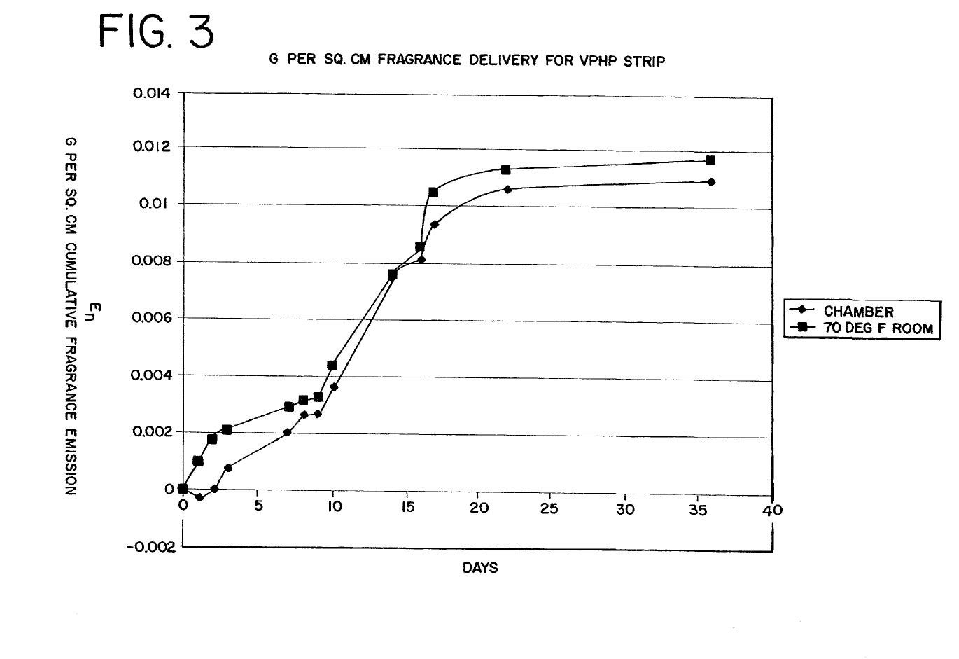

In other embodiments, such as illustrated in FIGS. 1 and 3, the fragrance is delivered at a relatively constant rate, which is suitable for open spaces where constant circulation of air establishes a relatively stable distribution of fragrance. The near steady-state delivery of FIGS. 1 and 3 extends for about a 22-day period.

To evaluate the perfume delivery characteristics of the substrate, a sample substrate is placed in a test chamber with a top opening that allows communication between the chamber and the atmosphere. The weight of the test chamber containing the substrate is recorded at predetermined intervals during a 36-day experiment period. As the fragrance is released from the copolymer matrix of the substrate, the weight of the substrate decreases and the weight loss is attributable to the released fragrance.

It is contemplated that the fragrance delivery characteristics are dependent on the total surface area of the sample. To minimize the effect of variation in the total surface area on the fragrance delivery characteristics, each sample has a planar dimension of about 1.27×9.525 cm². Because the thickness of the samples are in the range of from about 0.038 to about 0.051 cm, which is insignificant compared to the planar dimension of the samples, the total surface area of each sample remains substantially constant for all samples (1.27×9.525=12.1 cm²).

Various fragrances and substrate or strip thicknesses used in the fragrance delivery test are listed in Table 1.

TABLE 1

| | Description |
|---|---|
| | Scent type, % level fragrance, |
| Sample # | thickness of strip |
| 1 | Scent B MOD1 @ 20%, 15 mil |
| 2 | Scent B MOD1 @ 20%, 20 mil |
| 3 | Scent C MOD1 @ 20%, 15 mil |
| 4 | Scent C MOD1 @ 20%, 20 mil |
| 5 | Scent B MOD2 @ 30%, 20 mil |
| 6 | Scent B MOD2 @ 30%, 15 mil |
| 7 | Scent C MOD2 @ 30%, 15 mil |
| 8 | Scent C MOD2 @ 30%, 20 mil |
| 9 | MOD4 @ 30%, 20 mil |
| 10 | MOD4 @ 30%, 15 mil |

The recorded and averaged data is graphically illustrated in FIGS. 1-4. FIGS. 1-2 graphically illustrate cumulative fragrance depletion Dₙ versus time for samples 8 and 9 of Table 1 and FIGS. 3-4 graphically illustrate a normalized cumulative fragrance emission Eₙ per unit surface area for samples 8 and 9 of Table 1.

Turning first to FIGS. 1-2, the cumulative fragrance depletion Dₙ can be defined as follows:

Dₙ=((Pₙ−P₀)/P₀)×100,

where Pₙ is the current weight of the sample (on day n) and P₀ is the weight of the sample on day 0. As each experiment begins immediately after preparation of the sample, it is assumed that only an insignificant amount of fragrance is lost during the preparation and that the fragrance impregnated in the sample at the beginning of the experiment it is about equal the amount of the fragrance incorporated in the sample during the manufacturing process.

According to FIGS. 1-2, the time required for the substrate to achieve about 50% cumulative fragrance depletion is from about 5 to about 40, preferably from about 5 to about 30, more preferably from about 10 to about 30, and mostly preferably from about 15 to about 25, days. Moreover, the time required for the substrate to achieve about 33.3% cumulative perfume depletion is from about 2 to about 30, preferably from about 2 to about 20, more preferably from about 2 to about 15, and mostly preferably from about 2 to about 10, days. Finally, the time required for the substrate to achieve about 25% cumulative perfume depletion is from about 1 to about 30, preferably from about 1 to about 20, more preferably from about 1 to about 15, and most preferably from about 1 to about 10, days.

Turning to FIGS. 3-4, the normalized Eₙ (g/cm²) can be defined as follows:

Eₙ=(Pₙ−P₀)/S,

where S is the surface area through which the fragrance is emitted. As stated above, because the thickness of the samples are in the range of from about 0.038 to about 0.051 cm, which is insignificant compared to the planar dimension (1.27 cm×9.525 cm=12.1 cm²) of the samples, S remains substantially constant for all samples, i.e. ˜12.1 cm².

According to FIGS. 3-4, the time required for the substrate to achieve about 0.008 g/cm² cumulative perfume emission is from about 5 to about 40, preferably from about 5 to about 30, more preferably from about 10 to about 30, and mostly preferably from about 15 to about 25 days. Moreover, the time required for the substrate to achieve about 0.006 g/cm² cumulative perfume emission is from about 2 to about 30, preferably from about 3 to about 20, more preferably from about 3 to about 15, and mostly preferably from about 3 to about 13, days. Finally, the time required for the substrate to achieve about 0.004 g/cm² cumulative perfume emission is from about 1 to about 30, preferably from about 1 to about 20, more preferably from about 1 to about 15, and most preferably from about 1 to about 12, days.

In order to evaluate the accuracy of the fragrance delivery, experiments were carried out in a chamber and a parallel experiment was carried out in a 70° F. room. The weight of the sample was measured and recorded at identical predetermined intervals. A fragrance depletion curve is then obtained by similarly plotting the cumulative fragrance depletion (Dₙ) versus time. In FIGS. 1 and 3, fragrance depletion/emission curves for both the test chamber experiment and parallel room experiment are plotted together. FIGS. 1 and 3 clearly demonstrate that the two curves are in reasonable agreement with each other, and therefore that the fragrance delivery experiment in the smaller test chamber simulates the fragrance delivery of the substrate in an everyday application, such as a larger room, with sufficient accuracy.

Table 2 below illustrates an additional experiment where detection of a mold and mildew (M/M) odor was evaluated by a consumer panel consisting of a plurality of consumers. In the experiment, a consumer was asked to evaluate the detection of M/M odor in three chambers: a first chamber containing M/M odor only; a second chamber containing M/M odor and a control EVA strip without fragrance; and a third chamber containing M/M odor and one of the disclosed EVA strip impregnated with fragrance. As indicated in Table 2, the disclosed strip provides effective malodorant function for M/M odor for at least 30 days.

TABLE 2

| | Mean Strength of Mold & Mildew | | | | |
|---|---|---|---|---|---|
| | (0 = Do not detect any malodor, 7 = Very | | | | |
| | strong malodor) | | | | |
| | | | | Day 22 (Added | |
| | | | | M/M to all | |
| | | | Day 21 | samples after | Day 30 |
| | | | (Same | test on Day 21. | (Same |
| | Day 1 | Day 2 | samples | Retest 24 hours | samples |
| | (0 | (24 | as | after M/M is | as |
| | Hour) | Hours) | Day 1) | added) | Day 22) |
| Mold & Mildew | Prepare | 4.13 | 5.42 | 4.50 | 3.13 |
| (M/M) only | Samples | | | | |
| M/M + EVA strip | Prepare | 2.73 | 3.92 | 3.81 | 2.40 |
| w/o fragrance | Samples | | | | |
| M/M + EVA strip | Prepare | 0.87 | 1.50 | 1.94 | 1.40 |
| w/fragrance | Samples | | | | |

It is to be understood that the above disclosed embodiments are simply examples of the fragrance delivery characteristics that may be achieved by the disclosed substrate, and are not intended to limit the scope of the present disclosure. Modification of the embodiments, including for example modification of the composition and/or manufacturing process of the substrate, to achieve the disclosed or other suitable fragrance delivery characteristics would be apparent to one of ordinary skill in the art.

1. A substrate in a form of a transparent sheet comprising:
a copolymer of an olefin and at least one olefin derivative; and
a fragrance,
wherein the substrate is substantially free of homopolymeric material,
wherein the substrate has a thickness of from about 10 to about 50 mils, and
wherein the time required for the substrate to achieve about 50% cumulative fragrance depletion ranges from about 5 to about 40 days.

2. The substrate according to claim 1 wherein the olefin is selected from the group consisting of ethylene, propylene, and 1-butene.

3. The substrate according to claim 1 wherein the olefin derivative is selected from the group consisting of vinyl esters of saturated carboxylic acid, propenyl esters of saturated carboxylic acid, olefinically unsaturated organic acids and esters, and mixtures thereof.

4. The substrate according to claim 1 wherein the copolymer is selected from the group consisting of ethylene-vinyl acetate, propylene vinyl acetate, and a mixture thereof.

5. The substrate according to claim 1 wherein the fragrance is present in an amount ranging from about 1 to about 50 wt %.

6. The substrate according to claim 1 wherein the fragrance is present in an amount ranging from about 20 to about 30 wt %.

7. The substrate according to claim 1 wherein the time required for the substrate to achieve about 50% cumulative fragrance depletion ranges from about 15 to about 25 days.

8. The substrate according to claim 1 wherein the time required for the substrate to achieve about 33.3% cumulative fragrance depletion ranges from about 2 to about 10 days.

9. The substrate according to claim 1 wherein the time required for the substrate to achieve about 25% cumulative fragrance depletion ranges from about 1 to about 10 days.

10. (canceled)

11. A substrate in a form of a transparent sheet comprising:
a copolymer of an olefin and at least one olefin derivative; and
a fragrance,
wherein the substrate is substantially free of homopolymeric material,
wherein the substrate has a thickness of from about 10 to about 50 mils, and
wherein the time required for the substrate to achieve about 0.008 g/cm² cumulative fragrance emission ranges from about 5 to about 40 days.

12. The substrate according to claim 11 wherein the copolymer is selected from the group consisting of ethylene-vinyl acetate, propylene vinyl acetate, and a mixture thereof.

13. The substrate according to claim 11 wherein the fragrance is present in an amount ranging from about 1 to about 50 wt %.

14. The substrate according to claim 13 wherein the fragrance is present in an amount ranging from about 20 to about 30 wt %.

15. The substrate according to claim 11 wherein the time required for the substrate to achieve about 0.008 g/cm² cumulative fragrance emission ranges from about 15 to about 25 days.

16. The substrate according to claim 11 wherein the time required for the substrate to achieve about 0.006 g/cm² cumulative fragrance emission ranges from about 3 to about 13 days.

17. The substrate according to claim 11 wherein the time required for the substrate to achieve about 0.004 g/cm² cumulative fragrance emission ranges from about 1 to about 10 days.

18. A substrate in a form of a transparent sheet comprising:
a copolymer of an olefin and at least one olefin derivative selected from the group consisting of vinyl esters of saturated carboxylic acid, propenyl esters of saturated carboxylic acid, olefinically unsaturated organic acids and esters, and mixtures thereof; and
a fragrance,
wherein the substrate is substantially free of homopolymeric material;
wherein the substrate has a thickness of from about 10 to about 50 mils;
wherein the time required for the substrate to achieve about 50% cumulative fragrance depletion ranges from about 5 to about 40 days; and
wherein the time required for the substrate to achieve about 0.008 g/cm² cumulative fragrance emission ranges from about 5 to about 40 days.

19. The substrate according to claim 18 wherein the copolymer is ethylene-vinyl acetate.

20. The substrate according to claim 18 wherein the fragrance is present in an amount ranging from about 1 to about 50 wt %.
